(19) United States
(12) Patent Application Publication
(10) Pub. No.: US 2006/0041212 A1
(43) Pub. Date: Feb. 23, 2006

(54) Arthropometric method of diagnosing endocrine activity of a subject

(76) Inventor: Raymond C. Forbes, Pittsburgh, PA (US)

Correspondence Address: CLIFFORD A. POFF, 9800B MCKNIGHT ROAD, SUITE 115, PITTSBURGH, PA, 15237, US

(21) Appl. No.: 10/924,503

(22) Filed: Aug. 23, 2004

(51) Int. Cl. A61F 13/00

(52) U.S. Cl. 602/48; 600/300

(57) ABSTRACT

A method of measurement to determine inherited endocrine glandular pattern including but not limited to posterior pituitary gland deficiency and the use of a combination of oxytocin and vasopressin, to produce synthetic posterior pituitary gland. An adhesive bearing substrate is used to establish five sites at successive quarter points for circumferential length measurements along each of a forearm and lower leg of a subject. The measurements are used to calculate an ordered set of five ratios which are compared with the numerical values of an ordered set of five reference standards to determine hypo, normal or hyper activity of each of the ordered set of endocrine activity consisting of sex hormones; thyroid gland; posterior pituitary gland; anterior pituitary gland; and sex hormones.

### CROSS-REFERENCE TO RELATED APPLICATIONS

Not applicable

### BACKGROUND OF THE INVENTION

1. Field of the Invention

The present invention relates to a method of diagnosing endocrine activity of a human subject by using an adhesive bearing substrate to establish five sites along each of a forearm and lower leg for calculating an ordered set of five ratios and comparing with an ordered set of five reference standards to determine hypo, normal or hyper activity of each of the ordered set of endocrine activity, consisting of sex hormones; thyroid gland; posterior pituitary gland; anterior pituitary gland; and sex hormones. More particularly, the present invention uses an adhesive bearing substrate to derive circumferential measurements at each of the established five spaced and consecutive sites along each of a forearm and lower leg and then calculating a ratio of each successive lower leg circumferential length measurement divided by the respective forearm circumferential length measurement.

2. Description of the Prior Art

It is well known in the art that the pituitary gland located at the base of the brain has two distinct parts, the anterior and the posterior lobes, each of which releases different hormones which affect bone growth and regulate activity in other glands. It is not well known that posterior pituitary gland deficiency exists in a significant number of individuals. Such deficiencies are not usually detected or treated. The posterior pituitary gland of the pituitary body is primarily glandular in nature and produces oxytocin and vasopressin as the principal chemical substances. A posterior pituitary gland deficiency can manifest as certain external physical characteristics in humans. U.S. Pat. No. 6,333,333 discloses the use of oxytocin alone or in combination with other substances to treat certain conditions as, for example, bone disorders. However, it is believed unknown to use the combination of oxytocin and vasopressin to form a synthetic posterior pituitary gland for the treatment of bone disorders and other diseases. A thyroid deficiency is well known to exist in the human patient population. An external physical characteristic of thyroid deficiency is a thickened enlargement of the ankles.

Melvin E. Page, D.D.S, disclosed in Degeneration/Regeneration Published by Nutritional Development (copyright 1949) a system of body measurements used to diagnose such a deficiency. The measurements are taken by a second party using a tape measure, which is in inches that are divided into tenths. The measurements consist of circumferential measurements of the arms and legs at five equidistant points on each limb. There is an arm circumference at the wrist level, another at one-fourth the distance from the wrist to the elbow, another at one-half the distance from the wrist to the elbow, another at three-fourths the distance from the wrist to the elbow, and another at the elbow. They also consist of an ankle circumference, a one-fourth circumference from the ankle to the knee, a one-half circumference, a three-fourths circumference, and a knee circumference. The wrist circumference is divided into the ankle circumference, the ¼ arm circumference is divided into the ¼ leg circumference, the ½ arm circumference is divided into the ½ leg circumference, the ¾ arm circumference is divided into the ¾ leg circumference, and the elbow circumference is divided into the knee circumference. These quotients are representative of the activity of different endocrine glands.

The quotients were plotted in a graph developed to depict glandular patterns with the measurements being obtained by a second party. The norms for the graph appear as a family of base line norms and were established by Dr. Page after measuring and blood testing thousands of patients. Duplicate graphs are used to depict glandular patterns based on empirical values for the right side and for the left side of an individual, respectively. The normal empirical reference values of the limbs at the right side of male and female are plotted along the Y-axis of a first graph and normal empirical reference values of the limbs at the left side of male and female are plotted along the Y-axis of a second graph. In each of the graphs, spaced along the X axis is vertical division line to plot a value for the sex gland; a vertical division line to a plot value for the thyroid gland; a vertical division line to plot a value for the posterior pituitary gland; a vertical division line to plot a value for the anterior pituitary gland; and a vertical division line to plot a value for a measure of activity of the sex glands. The normal empirical reference values vary in magnitude, thus giving the base line graphs a saw tooth waveform along the X-axis, which can lead to user confusion.

Using a reference point, which consists of the wrist and ankle quotient, normal reference values are plotted or determined for each individual. Deviation from this reference norm can indicate hyper or hypo activity of the thyroid gland, the posterior pituitary gland, the anterior pituitary gland, the adrenal cortex, plus the degree of activity of the sex glands, which produce estrogen and testosterone. Dr. Page estimated that 88 percent of females and 39 percent of males have some degree of deficiency of the posterior pituitary gland. The body measurements although obtained by a second party have proven to be a quick diagnostic determinant of endocrine gland activity and can be used to determine the fastest and most accurate treatment using hormones or glandular supplements. It also provides new diagnostic information that is not available by current diagnostic methods. For example, the thyroid gland is stimulated by the anterior pituitary gland and this gland is often hypofunctioning along with the thyroid. The preferred treatment sequence with this new information is to give anterior pituitary glandular substance first and then the thyroid. Sometimes anterior pituitary only is needed and thyroid medication is not required since the thyroid becomes better stimulated by TSH (thyroid stimulating hormone) coming from the anterior pituitary gland.

Many diabetes cases have an associated posterior pituitary gland deficiency and/or an over active anterior pituitary gland. This is not normally diagnosed or treated. The measurements permit such a diagnosis. When these glands are treated first, insulin or other hypoglycemic drugs are often not required or they are needed in much smaller dosages. An under active posterior pituitary gland allows the adrenal cortex to over function producing high blood pressure in some cases and exaggerated secondary sex characteristics such as unwanted facial hair in women. Posterior pituitary gland supplementation improves or eliminates these problems due to the feedback effect on the adrenal cortex. Page postulated that spontaneous abortion and difficulty in becoming pregnant in some women is due to posterior pituitary gland hypo function. He found that supplementation with posterior pituitary gland improves or eliminates this problem. Page also found that 95% of all cancer patients have an overactive anterior pituitary gland. Suppression on this gland with sex hormones and/or insulin has been used as an adjunctive treatment for cancer. The measurements allow for this over activity to be diagnosed and can indicate which sex hormones to use in treatment. Under activity of the anterior pituitary gland often causes hypoglycemia. It also causes weight problems and lack of sufficient stimulation of the thyroid gland. When under activity of the anterior pituitary gland is combined with under activity of the posterior pituitary gland the weight problems are worse. Excessive gynicity (too many female hormones) or andricity (too many male hormones) is also associated with some cancers. The empirical values obtained by physical measurements enable this diagnosis and treatment using the proper sex hormones. Furthermore, such a diagnosis enables the proper choice of sex hormones in the treatment of pre and postmenopausal problems in women. It also allows for correction of the andric/gynic balance in both males and females before or after menopause in women or the male climacteric.

Since Page measured thousands of patients, he was able to identify various diseases and disease trends with various glandular patterns. Treatment with glandular substances or hormones provides alleviation or cure of many of the diseases associated with each glandular pattern. It is not desirable to have excess under activity (hypofunction) or over activity (hyperfunction) of any gland. Calcium/phosphorous ratios on a controlled diet for three days and after a twelve hour fast are used to determine the correct dosages of glandular substances or hormones. In some instances, the feedback effect of opposing glands is used to suppress glandular hyperactivity. The chart below describes the associations that Page was able to make with various glandular patterns and physical characteristics and various diseases. One or more of these glandular patterns is usually found in approximately 80% of the population. It takes only minute amounts of glandular supplements or hormones to correct the problem. If left untreated serious degenerative diseases and other problems can result.

| | POSSIBLE ASSOCIATED |
|---|---|
| GLANDULAR | CHARACTERISTICS AND |
| PATTERN | PROBLEMS |
| Hpo-anterior Pituitary | usually fat |
| (Under active) | spare tire |
| | large appetite |
| | craving for sweets |
| | low blood sugar |
| Hyper-anterior pituitary | duodenal ulcer |
| (Overactive) | Cancer |
| | heart trouble |
| | Diabetes |
| | great drive - especially in men |
| | hair loss forward and top of head (men) |
| | Pyorrhea |
| Hypo-posterior pituitary | weight primarily in thighs |
| (Under active) | women-double hip curve |
| | enlarged calf - midway between the ankle and |
| | knee |
| | men-double chin/potbelly |
| | baldness crown of head -- men |
| | frequent urination |
| | diabetes insipidus |
| | Hypertension |
| | Pyorrhea |
| | alcoholism |
| | depression |
| | manic depression |
| | Cataracts |
| | dental decay |
| | spontaneous abortion |
| | Arthritis |
| Hyper-posterior pituitary | decreased adrenal cortex function (darkened |
| (Overactive) | skin) |
| | decreased ability to handle stress |
| Hyperthyroid | weak heart |
| (Overactive) | Angina |
| | coronary occlusion |
| | Diarrhea |
| | hemorrhoids (tightened sphincter) |
| | mentally alert but possibly unstable |
| | fits of genius - would not make a good judge |
| | hairline recedes over each eye |
| | may or may not have increased metabolic rate |
| | increased metabolic rate is more apt in toxic |
| | thyroid |
| | flushes in women at climacteric due to |
| | vasomotor instability |
| | may be explosive emotionally |
| Hypothyroid | thick legs above the ankle |
| (Under active) | horizontal wrinkles on the forehead - a look of |
| | strain and fatigue |
| | some are fat but most are thin |
| | Pyorrhea |
| | always tired |
| Gynic (increased | usually a follower more than a leader |
| female hormones) | (mental ability may overcome this) |
| | extreme gynic (gentle) |
| | susceptible to cardiac insufficiency later in life |
| | sympathetic, retiring in nature |
| | interests often lie in home and children |
| | enjoys social functions, music, fine arts, often |
| | creative and sensitive |
| | intuitive thinker |
| | breast cancer |
| | schizophrenia |
| | lung cancer |
| | liver cancer |
| | intestinal cancer |
| Andric (increased | may be coarse |
| male hormones) | dominating in manner |
| | usually ambitious, aggressive |
| | logical thinker, leaders in chosen fields |
| | make major decisions often without consulting |
| | wife |
| | like to dominate all situations |
| | duodenal ulcer |
| | prostate cancer |
| | heart problems |
| | high blood pressure |

By looking at the key, you can see the various glandular patterns and the various combinations and problems that can exist. The patient can be either andric or gynic. Some people are right in the middle. If the patient is andric, he or she may require estrogen. If the patient is gynic he or she may require testosterone. If the patient is right in the middle, he or she may require a combination of both estrogen and testosterone. In addition, the patient can be hypo posterior pituitary, or hyper posterior pituitary, or hypo anterior and posterior pituitary, or hyper anterior and posterior pituitary. In addition, the patient can be hypo thyroid or hyperthyroid. Many possible combinations exist and without the empirical reference values, it is extremely difficult and time consuming to diagnose and treat a patient. In the matter of treating the patient, the empirical reference values are used to establish the choice of supplement or supplements to correct problems while monitoring the blood chemistries on a controlled diet for three days and after a twelve hour fast, checking the calcium/phosphorus ratio to determine the correct dosages. Without the measurements and without monitoring calcium/phosphorous ratios any correction is extremely haphazard and time consuming. The dosage or dosages that provide the ideal calcium/phosphorous ratio are used as the maintenance dosage and the ratios checked periodically, usually on a yearly basis.

External physical signs can also be used as an indicator of posterior pituitary gland under activity. They are baldness of the crown of the head in men who also have the baldness gene, a double chin in men, a double hip curve in women, and an exadurated curvature of the lower leg half way between the knee and ankle in both men and women. A specific gravity of the urine below 1.022 on a diet free of caffeine, alcohol, drugs, or medications is also an indicator of under activity of the posterior pituitary gland. This is because ADH or antidiuretic hormone (vasopressin) comes from this gland and when the gland is under functioning there is frequency of urination and the specific gravity of the urine is low. By blood testing 25 young healthy nurses on a good diet with no infection or disease Dr Page found that their serum calcium/phosphorous ratio was 2½:1 i.e. if the calcium was 10 the phosphorous was 4. He also observed diabetic patients on strictly controlled diets in a hospital setting and with their blood sugar being held at ideal levels and found the serum calcium and phosphorous ratio to be the same. 2½:1. He also confirmed his findings with over 40.000 blood chemistries on several thousand patients. The U.S. Air Force department of dental research also confirmed this to be the correct ratio of serum calcium to phosphorous. Page also observed that when there was infection present or an endocrine imbalance this ratio would alter. This ratio on a controlled diet for three days and after a twelve hour fast became his tool to monitor endocrine gland supplementation. A calcium/phosphorous ratio of 2½:1 represents an ideal endocrine glandular balance. He found this ratio to be quite sensitive to extremely small amounts of glandular supplements, as small as 1/1200 mg of estrogen and 1/800 mg of pituitary supplementation, and 1/50 grain of thyroid. The ratio can be used as an extremely sensitive tool to determine endocrine gland dosages. Dosages are administered at the smallest effective dosage first and are gradually increased as necessary using calcium/phosphorous and blood sugar values, plus blood pressure, and the specific gravity of the urine as a guide.

One glandular pattern detected by the measurements is posterior pituitary gland hypo activity or hyperactivity. This invention more specifically relates to hypo activity of this gland. Measurements of thousands of patients in the U.S. have revealed that 88% of females and 39% of males have some sub function of this gland. In current medical practice, this deficiency is not treated at all unless there is extreme under activity of the gland usually due to some trauma. Natural posterior pituitary glandular substance derived from cattle has been used to treat the less severe deficiencies, which nevertheless cause severe problems. These problems can be diabetes insipidus, depression, manic depression, some forms of high blood pressure, variations of the calcium/phosphorous ratio leading to degenerative arthritis, osteoporosis, periodontal disease, and excessive tooth decay. Dosages of posterior pituitary gland can be adjusted by monitoring serum calcium and phosphorous values on a controlled diet for at least three days with the testing dosage being taken each day and a blood sample taken after the third day and after a twelve hour fast. The controlled diet is as follows: Meat, fish, eggs, cooked any way, vegetables raw or cooked, oil and vinegar on salads, nuts, and popcorn if desired but no peanuts or honey, no foods containing sweet or white flour, no sugar, no milk, no cheese, no fruit, no alcohol, no fruit or vegetable juices, no coffee, no drinks with caffeine. The final maintenance dosage is the one that produces the ideal calcium/phosphorous ratio.

Specific gravity of the urine on the controlled diet for three days can also be used to help determine dosages since the antidiuretic hormone (vasopressin) comes from the posterior pituitary gland and a deficiency of the posterior pituitary gland produces urine specific gravities, which are abnormal. The dosage that produces specific gravity values at or close to 1.022 is the preferred dosage. The need to treat with posterior pituitary substance can be determined with body measurements. Clinical signs of the deficiency are a double hip curve in women, bald spot at the crown of the head in some men, a double chin in men, abnormal serum calcium and phosphorous values on a controlled diet for three days, abnormal urine specific gravity values, and sometimes physical symptoms and diseases such as diabetes insipidus, high blood pressure, frequency of urination, depression and manic depression, periodontal disease, osteoporosis and arthritis.

Posterior pituitary glandular supplement works in several ways. One way is due to its feedback effect on the adrenal cortex. By this mechanism, it prevents the adrenal cortex from over functioning producing high cortisol levels. High plasma cortisol has been implicated in depression and U.S. Pat. No. 4,814,333 describes a drug, which blocks cortisol synthesis in the adrenal cortex and produces favorable results with depression. By supplementing the posterior pituitary gland to its normal level of activity, this drug is not required. The feedback effect of the posterior pituitary gland keeps the cortisol levels in balance. This drug has produced liver problems and such a problem does not occur with posterior pituitary supplementation. Posterior pituitary gland supplementation has been used successfully in the treatment of depression and manic depression with no side effects. Because it has no deleterious effect on the liver, it can be used for treatment of depression in patients with liver damage due to drug over dosage or alcoholism. Additionally, antidiuretic hormone, ADH comes from the posterior pituitary gland. When this gland sub functions, it can cause diabetes insipidus due to the elimination of large amounts urine. Posterior pituitary restores natural function to this gland thereby improving or curing diabetes insipidus. It can also be used as a treatment for excessive frequency of urination. Page also found that many of his women patients were more successful in becoming pregnant with posterior pituitary gland supplementation. When the adrenal cortex is over functioning, it can also cause increased blood pressure. Restoring natural function to the posterior pituitary gland improves blood pressure with no side effects such as dry mouth that often occurs with most blood pressure medication.

Another benefit of this posterior pituitary substance is to produce more favorable calcium/phosphorous ratios. The effect of this glandular substance on calcium phosphorous ratios improves conditions where there is excess calcium or decreased available calcium in the system. These conditions can produce such diseases as arteriosclerosis, cataracts, kidney stones, bone spurs, degenerative arthritis, increased dental decay, osteoporosis, and periodontal disease.

Posterior pituitary gland deficiency can also cause weight problems. When combined with anterior pituitary gland deficiency the weight problems are worse. Posterior pituitary gland deficiency causes a double chin and a potbelly in men. It causes a double hip curve or “saddle bags” in women. Anterior pituitary gland deficiency causes a spare tire of fat in the midsection of both men and women. Synthetic posterior pituitary gland can be used as a weight reduction or weight control supplement if the need for it is determined with body measurements and/or blood tests and checks of urine specific gravity. It can also be combined with natural anterior pituitary if the patient is deficient in whole pituitary as determined by body measurements and/or blood tests. In this system of glandular correction, the worst glandular deficiency or excess is treated first and the calcium and phosphorous ratios are checked after the patients are on the controlled diet for three days and taking the designated supplement. Subsequent dosages are added and various supplements are tried until the ideal combination is achieved. This combination is that at which the calcium/phosphorous ratio is optimal. The system is essentially a bioassay.

### SUMMARY OF THE INVENTION

In accordance with the present invention there is provided an anthropometric method of diagnosing endocrine activity of a subject, the method including the steps of using an adhesive bearing substrate to establish five sites for circumferential length measurement along each of a forearm and lower leg of a subject, each of the five sites being established by indicia on the substrate at successive quarter points commencing at the outer most site of each of the forearm and lower leg of a subject, calculating an ordered set of five ratios comprising the quotient of each successive lower leg circumferential length measurement divided by the respective forearm circumferential length measurement for each circumferential length measurement at the quarter points, and comparing the numerical values of the ordered set of five ratios to numerical values of an ordered set of five reference standards to determine hypo, normal or hyper activity of each of the ordered set of endocrine activity consisting of sex hormones; thyroid gland; posterior pituitary gland; anterior pituitary gland; and sex hormones.

The anthropometric method according to the invention preferably provides that the five sites for circumferential length measurement along each of a forearm and lower leg of a subject are further defined to comprise: an arm circumference at the wrist level; an arm circumference at one fourth the distance from the wrist to the elbow; an arm circumference at one half the distance from the wrist to the elbow; an arm circumference at three fourths the distance from the wrist to the elbow; an arm circumference at the elbow; a lower leg circumference at an ankle level; a lower leg circumference at one fourth the distance from the ankle to the knee; lower leg circumference at a one half the distance ankle to the knee; a lower leg circumference at three fourths the distance from the ankle to the knee; and lower leg circumference at the knee circumference.

The measurement according to the method of the present invention using the adhesive bearing substrate to establish five sites for circumferential length measurement along the forearm of a subject includes seating the subject in a chair with the subjects feet flat on the floor and the chosen arm bent slightly with the palm of the hand placed on the knee, applying an elbow indicia mark to the center of the elbow and a wrist indicia mark to the center of the wrist at the site of a protruding bone at the outside of the arm, arranging the substrate on the outside of the arm to span the distance between the elbow indicia mark and the wrist indicia mark, recording the elbow indicia mark and the wrist indicia mark on the substrate, using length graduations of a rule to establish and then apply indicia markings at each of four equal length sections between the elbow indicia mark and the wrist indicia mark along the substrate, and adhesively attaching the substrate to a subject to span the distance between the elbow indicia mark and the wrist indicia mark to display the indicia markings of four equal length sections.

The measurement according to the method of the present invention using the adhesive bearing substrate to establish five sites for circumferential length measurement along the lower leg of a subject includes, seating the subject in a chair with the subject's heel on the floor and the bottom flat surface of the foot at right angles to the long axis of the lower leg, applying a lower leg indicia marks to the center of the knee cap and an ankle bone mark on the outside of the ankle in the middle of the protruding bone, arranging the substrate on the outside of the lower leg to span the distance between the indicia mark at the center of the knee cap and the ankle bone mark on the outside of the ankle, recording the lower leg indicia marks on the substrate, using length graduations of a rule to establish and then apply lower leg indicia markings at each of four equal length sections along the substrate, and adhesively attaching the substrate to a subject to span the distance between the lower leg indicia marks to display the indicia markings of four equal length sections.

### BRIEF DESCRIPTION OF THE SEVERAL VIEWS OF THE DRAWINGS

The present invention will be more fully understood when the following description is read in light of the accompanying drawings in which:

FIG. 1 is an illustration of the use of a substrate to establish measurement sites along the forearm of an individual;

FIG. 2 is an illustration of the use of a substrate to establish circumferential measurements at sites established in the illustration of FIG. 1;

FIG. 3 is an illustration of the use of a substrate to establish measurement sites along the lower leg of an individual; and

FIG. 4 is an illustration of the use of a substrate to establish circumferential measurements at sites established in the illustration of FIG. 3.

### DETAILED DESCRIPTION OF THE INVENTION

The present invention obviates the requirement for a second party such as a healthcare practitioner to determine the inherited glandular pattern of the patient. Empirical measurements are made by the use of an adhesive bearing substrate to establish five sites for circumferential length measurement along each of a forearm and a lower leg of a subject. The adhesive bearing substrate may take the form of adhesive tape or some form of sticky tape because it remains with great integrity at the placement site. The use of an adhesive bearing tape fulfills the need for reliable accuracy, which is usually not assured when using only a conventional tape measure. The accuracy of the empirical measurements is imperative for successful results of the invention. A ruler divided in inches with each inch subdivided into tenths is used to divide into fourths the measured lengths of an arm segment and a leg segment. The ruler is also used to determine the circumferential measurements. However, a ruler sheet or some other well known measuring instrument or measuring grid is also suitable to determine length measurement in inches to tenth of an inch. According to the present invention, it is not necessary to make measurements of both arms and legs of an individual to produce the empirical values and thus is not required to compute an average of the quotients when using an anthropometric analysis table or, if desired using graph of the type described by Dr. Page hereinbefore. When measurements of all limbs are made, the quotients for each side of the body are plotted in an anthropometric analysis table. If there is uncertainty in interpretation of the anthropometric analysis table plotting each side of the body then average quotients from both sides of the body may be plotted.

### Arm Measurements

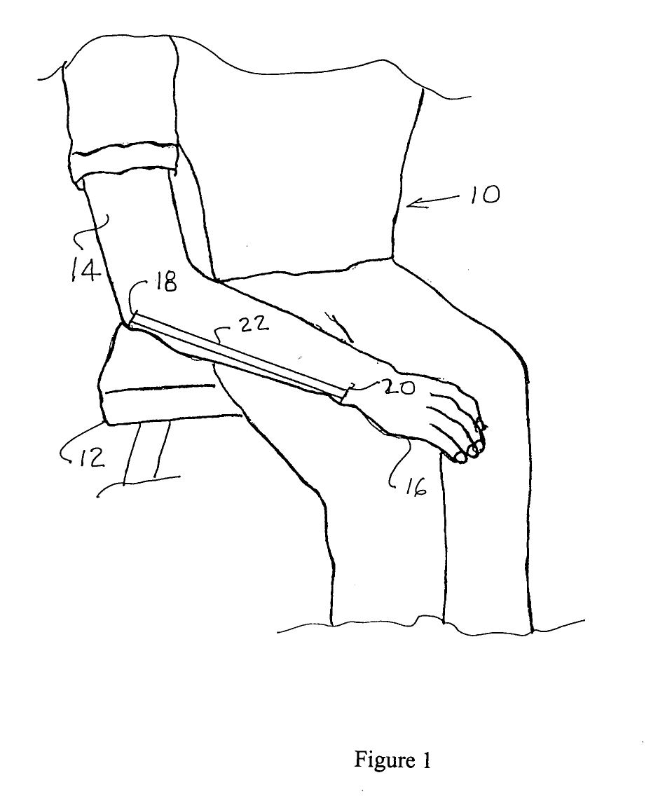

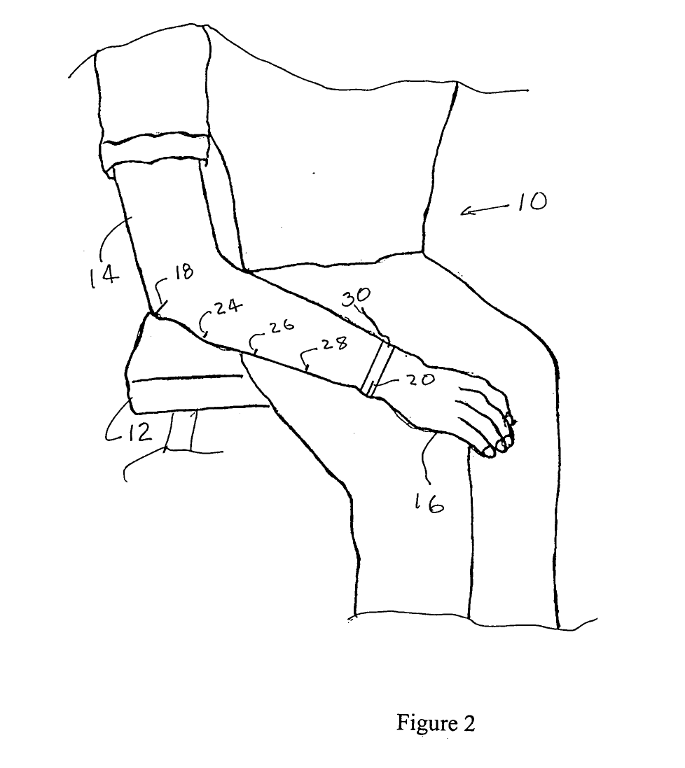

As shown in FIG. 1, a patient 10 is positioned in a chair 12 with the patient's feet, not shown, lying flat on the floor. The right arm 14 is bent slightly as shown and the palm of the hand 16 is placed and supported on the knee. Indicia 18 and 20 such as a thin line segment are applied with a pen to each of the outwardly directed surface at the center of the elbow and the center of the wrist where the bone protrudes on the outside. Adhesive bearing substrate 22 is applied to the outside of the arm to traverse the space between the indicia 18 and 20. It is preferable to use ½-inch wide medical grade adhesive tape that is not too sticky. Johnson and Johnson makes composite paper and cloth first aid tape that is particularly suitable. The substrate is cut to the exact length needed to traverse the distance between the indicia 18 and 20. The tape is placed on a ruler, per se well known in the art, and the exact length is recorded. The measured length is then divided by four to establish four equal segments of length at ¼, ½ and ¾ of the tape length. The length segments marked on the tape whereby the tape is divided into successive quarter points and placed on the forearm commencing at the outer most site of the forearm. As shown in FIG. 2, additional indicia 24, 26 and 28 are applied to the forearm at each of the pen marks at the ¼, ½, and ¾ lines on the tape, which is then removed from the arm. Lengths of tape 30, about 6 inches long, are then prepared for use. With the arm in the slightly bent position and resting on the knee, one length of tape 30 is placed around the wrist with care taken to pass the tape directly over the indicia 18. Overlap the tape and stick it to itself. The tape should be formed smoothly around the wrist. The tissue should rest naturally without pinching the tissue. Next, run the blade of scissors under the tape and cut it. Transfer the piece of tape to the ruler and record the exact length. Repeat this measuring procedure at each of the indicia 24, 26 and 28. The measuring procedures at the indicia 24, 26 and 28 differs by align the edge of tape so that the top edge of the tape which is nearest the body just touches the indicia 24, 26 and 28. The measurement process is repeated for establishing measurements for both of the forearms of the subject and a record of the circumferences in the respective measurement sites are recorded on a measurement chart given hereinafter.

### Leg Measurements

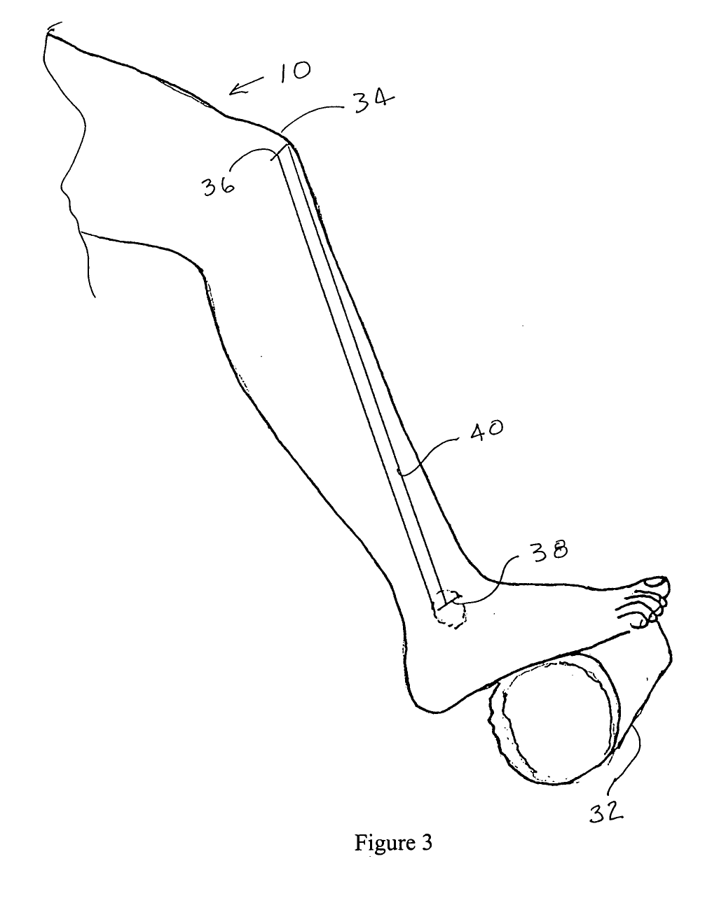

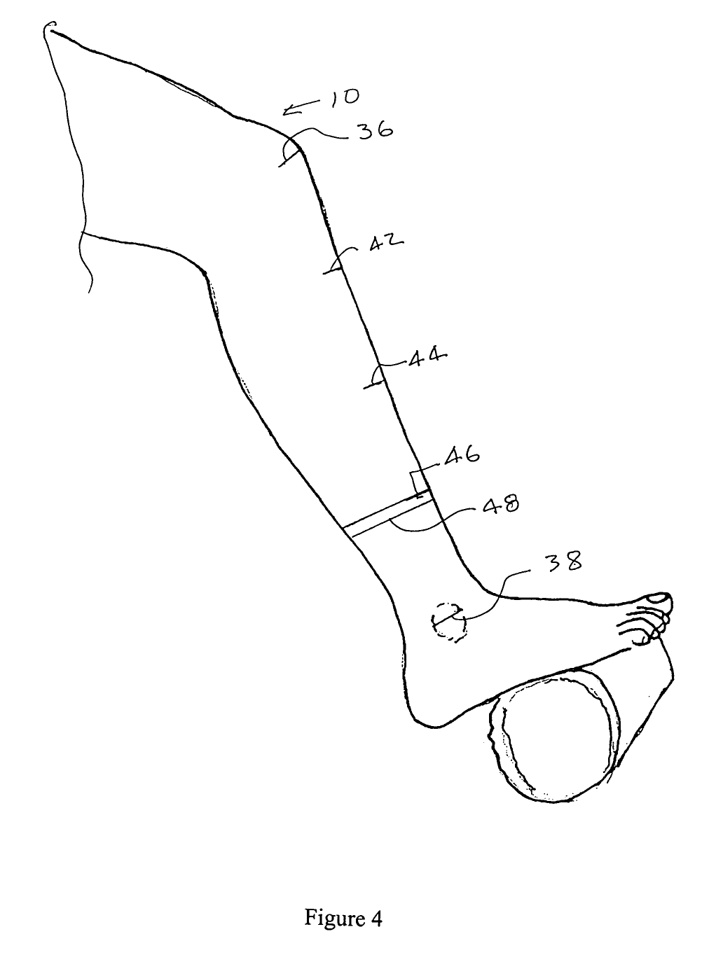

As shown in FIG. 3, the patient 10 is again positioned in a chair, not shown, with the patient's foot supported on a cylinder 32 in the mid portion of the foot. The cylinder is positioned on the floor such that the knee 34 is slightly bent, as shown, thus angling, the lower leg to the vertical. Indicia 36 and 38 such as a thin line segment is applied with a pen to the on the center of the kneecap and on the anklebone on the outside of the ankle in the middle of the protruding bone, respectively. Adhesive bearing substrate 40 is applied on the kneecap, the end of the tape touching the indicia 36, and then the substrate is stretch from this point to the anklebone where the substrate is cut so that the cut end meets the indicia 38 on the ankle. The cut length of substrate is placed on a ruler and the exact length is recorded. The measured length is then divided by four to establish four equal segments of length at ¼, ½, and ¾ of the substrate length. The length segments are marked on the tape whereby the tape is divided into successive quarter points and the marked tape is placed on the leg commencing at the outer most site on the ankle. As shown in FIG. 4, additional indicia 42, 44 and 46 is applied to the lower leg at each of the pen marks at the ¼, ½, and ¾ lines on the tape, which is then removed from the leg. Lengths of tape 48, about 10 inches long are then prepared for use to determine circumferences at the knee, at indicia 42, 44 and 46, and at the ankle. With the leg remaining in the slightly bent position, place the tape over the pen mark on the knee and the ankle. For the measurements at indicia 42 and 44, the tape measurements are accomplished by placing the top edges of the tapes on the pen marks. When making the circumference measurement at indicia 46 on the leg, place the bottom edge of the tape above the mark instead of below the mark. Overlap the tape for each measurement and stick the tape upon itself. The tape should be formed smoothly around the sites along the lower leg. The leg tissue should rest naturally without pinching the tissue. Next, run the blade of scissors under the tape and cut it. Transfer the piece of tape to the ruler and record the exact length. Repeat this measuring procedure at each of the indicia 42, 44 and 46. The measuring procedures at the indicia 42, 44 and 46 differs by align the edge of tape so that the top edge of the tape which is nearest the body just touches the indicia 42, 44 and 46. The measurement process is repeated for establishing measurements for both of the lower leg of the subject and a record of the circumferences in the respective measurement sites are recorded on a limb length chart, as follows.

Limb length chart

| | |
|---|---|
| Right side | Left side |

| | | | |
|---|---|---|---|
| Leg Length | Arm Length | Leg Length | Arm Length |
| ( ) | ( ) | ( ) | ( ) |

Quotients are created by dividing the leg measurements
by the arm measurements.

After recording the circumferential measurements in the appropriate places in a circumferential measurement and quotient chart given below, the quotients are computed and introduced in the appropriate places. It is sufficient to round off all quotients to two decimal places.

Circumferential Measurement and Quotient Chart

| Right side | | | Left side | | |
|---|---|---|---|---|---|
| Wrist | Ankle | Quotient | Wrist | Ankle | Quotient |
| Circumference | Circumference | Ankle Divided | Circumference | Circumference | Ankle Divided |
| | | By Wrist | | | By Wrist |
| | | ( ) A | | | ( ) A |
| ¼ Arm | ¼ Leg | Quotient | ¼ Arm | ¼ Leg | Quotient |
| Circumference | Circumference | ¼ leg divided | Circumference | Circumference | ¼ leg divided |
| | | by ¼ arm | | | by ¼ arm |
| | | ( ) B | | | ( ) B |
| ½ Arm | ½ Leg | Quotient | ½ Arm | ½ Leg | Quotient |
| Circumference | Circumference | ½ leg divided | Circumference | Circumference | ½ divided by |
| | | by ½ arm | | | ½ arm |
| | | ( ) C | | | ( ) C |
| ¾ Arm | ¾ Leg | Quotient | ¾ Arm | ¾ Leg | Quotient |
| Circumference | Circumference | ¾ leg divided | Circumference | Circumference | ¾ leg divided |
| | | by ¾ arm | | | by ¾ arm |
| | | ( ) D | | | ( ) D |
| Elbow | Knee | Quotient | Elbow | Knee | Quotient |
| Circumference | Circumference | Knee divided | Circumference | Circumference | Knee divided |
| | | by Elbow | | | by Elbow |
| | | ( ) E | | | ( ) E |

The quotients for the right side and left side for each of the established five sites for circumferential length measurements along each of the forearms and each of the lower legs of a subject are computed. These values are then used for determine the average quotients for each of the establish five sites of circumferential length measurement along each of a forearm and lower leg of the subject as follows. Add the left side ankle/wrist quotient to the right side wrist quotient and divide by 2. Then, add the left side ¼ leg/¼ arm quotient to the right side ¼ leg/¼ arm quotient and divide by 2. Add the left side ½ leg/½ arm quotient to the right side ½ leg/½ arm quotient and divide by 2. Add the left side ¾ leg/¾ arm quotient to the right side ¾ leg/¾ arm quotient and divide by 2. Add the left side knee quotient/elbow quotient to the right side knee quotient/elbow quotient and divide by 2. Round off all values to two decimal places and introduce the results to the Averaged measurement table below.

Averaged Quotient Chart

| | | | | |
|---|---|---|---|---|
| Quotient Right | Quotient Right | Quotient Right | Quotient Right | Quotient Right |
| Wrist and Leg | ¼ Arm and Leg | ½ Arm and Leg | ¾ Arm and Leg | Elbow and Knee |
| Plus Quotient | Plus Quotient | Plus Quotient | Plus Quotient | Plus Quotient Left |
| Left Wrist and | Left ¼ Arm and | Left ½ Arm and | Left ¾ Arm and | Elbow and Knee |
| Leg Divided By | Leg Divided By | Leg Divided By | Leg Divided By | Divided By |
| 2 = | 2 = | 2 = | 2 = | 2 = |
| \overline{(A Value)} | \overline{(B Value)} | \overline{(C Value)} | \overline{(D Value)} | \overline{(E Value)} |

Two systems to analyze the results of these quotients are possible. The preferred system is to use the Anthropometric Analysis Table of the present invention and given below. The Anthropometric Analysis Table is a numerical table to define the glandular pattern of the patient. Such a table lends itself to easier interpretation of the measurement results, as compared to plot the quotients on in a saw tooth wave form of a graph taught by Dr. Page hereinbefore. Plotting in such a graph may be difficult and the use of the Anthropometric Analysis Table is simpler for an individual doing a self-assessment. The individual finds the quotient values, which can be averaged, in the respective columns of Measurement Table and the values circled for ease of comparison with the reference values all in the ordered sets of values.

Anthropometric Analysis Table

| | | Posterior | Anterior | |
|---|---|---|---|---|
| Sex | Thyroid | Pituitary | Pituitary | Sex |
| Hormones | Gland | Gland | Gland | Hormones |
| A Values | B Values | C Values | D Values | E Values |
| 2.05 | 1.80 | 1.88 | 1.84 | 2.05 |
| 2.04 | 1.79 | 1.87 | 1.83 | 2.04 |
| 2.03 | 1.78 | 1.86 | 1.82 | 2.03 |
| 2.02 | 1.77 | 1.85 | 1.81 | 2.02 |
| 2.01 | 1.76 | 1.84 | 1.80 | 2.01 |
| 2.00 | 1.75 | 1.83 | 1.79 | 2.00 |
| 1.99 | 1.74 | 1.82 | 1.78 | 1.99 |
| 1.98 | 1.73 | 1.81 | 1.77 | 1.98 |
| 1.97 | 1.72 | 1.80 | 1.76 | 1.97 |
| 1.96 | 1.71 | 1.79 | 1.75 | 1.96 |
| 1.95 | 1.70 | 1.78 | 1.74 | 1.95 |
| 1.94 | 1.69 | 1.77 | 1.73 | 1.94 |
| 1.93 | 1.68 | 1.76 | 1.72 | 1.93 |
| 1.92 | 1.67 | 1.75 | 1.71 | 1.92 |
| 1.91 | 1.66 | 1.74 | 1.70 | 1.91 |
| 1.90 | 1.65 | 1.73 | 1.69 | 1.90 |
| 1.89 | 1.64 | 1.72 | 1.68 | 1.89 |
| 1.88 | 1.63 | 1.71 | 1.67 | 1.88 |
| 1.87 | 1.62 | 1.70 | 1.66 | 1.87 |
| 1.86 | 1.61 | 1.69 | 1.65 | 1.86 |
| 1.85 | 1.60 | 1.68 | 1.64 | 1.85 |
| 1.84 | 1.59 | 1.67 | 1.63 | 1.84 |
| 1.83 | 1.58 | 1.66 | 1.62 | 1.83 |
| 1.82 | 1.57 | 1.65 | 1.61 | 1.82 |
| 1.81 | 1.56 | 1.64 | 1.60 | 1.81 |
| 1.80 | 1.55 | 1.63 | 1.59 | 1.80 |
| 1.79 | 1.54 | 1.62 | 1.58 | 1.79 |
| 1.78 | 1.53 | 1.61 | 1.57 | 1.78 |
| 1.77 | 1.52 | 1.60 | 1.56 | 1.77 |
| 1.76 | 1.51 | 1.59 | 1.55 | 1.76 |
| 1.75 | 1.50 | 1.58 | 1.54 | 1.75 |
| 1.74 | 1.49 | 1.57 | 1.53 | 1.74 |
| 1.73 | 1.48 | 1.56 | 1.52 | 1.73 |
| 1.72 | 1.47 | 1.55 | 1.51 | 1.72 |
| 1.71 | 1.46 | 1.54 | 1.50 | 1.71 |
| 1.70 | 1.45 | 1.53 | 1.49 | 1.70 |
| 1.69 | 1.44 | 1.52 | 1.48 | 1.69 |
| 1.68 | 1.43 | 1.51 | 1.47 | 1.68 |
| 1.67 | 1.42 | 1.50 | 1.46 | 1.67 |
| 1.66 | 1.41 | 1.49 | 1.45 | 1.66 |
| 1.65 | 1.40 | 1.48 | 1.44 | 1.65 |
| 1.64 | 1.39 | 1.47 | 1.43 | 1.64 |
| 1.63 | 1.38 | 1.46 | 1.42 | 1.63 |
| 1.62 | 1.37 | 1.45 | 1.41 | 1.62 |
| 1.61 | 1.36 | 1.44 | 1.40 | 1.61 |
| 1.60 | 1.35 | 1.43 | 1.39 | 1.60 |
| 1.59 | 1.34 | 1.42 | 1.38 | 1.59 |
| 1.58 | 1.33 | 1.41 | 1.37 | 1.58 |
| 1.57 | 1.32 | 1.40 | 1.36 | 1.57 |
| 1.56 | 1.31 | 1.39 | 1.35 | 1.56 |
| 1.55 (female | 1.30 | 1.38 | 1.34 | 1.55 |
| reference | | | | |
| point) | | | | |
| 1.54 | 1.29 | 1.37 | 1.33 | 1.54 |
| 1.53 | 1.28 | 1.36 | 1.32 | 1.53 |
| 1.52 | 1.27 | 1.35 | 1.31 | 1.52 |
| 1.51 | 1.26 | 1.34 | 1.30 | 1.51 |
| 1.50 | 1.25 | 1.33 | 1.29 | 1.50 |
| 1.49 | 1.24 | 1.32 | 1.28 | 1.49 |
| 1.48 (male | 1.23 | 1.31 | 1.27 | 1.48 |
| reference | | | | |
| point) | | | | |
| 1.47 | 1.22 | 1.30 | 1.26 | 1.47 |
| 1.46 | 1.21 | 1.29 | 1.25 | 1.46 |
| 1.45 | 1.20 | 1.28 | 1.24 | 1.45 |
| 1.44 | 1.19 | 1.27 | 1.23 | 1.44 |
| 1.43 | 1.18 | 1.26 | 1.22 | 1.43 |
| 1.42 | 1.17 | 1.25 | 1.21 | 1.42 |
| 1.41 | 1.16 | 1.24 | 1.20 | 1.41 |
| 1.40 | 1.15 | 1.23 | 1.19 | 1.40 |
| 1.39 | 1.14 | 1.22 | 1.18 | 1.39 |
| 1.38 | 1.13 | 1.21 | 1.17 | 1.38 |
| 1.37 | 1.12 | 1.20 | 1.16 | 1.37 |
| 1.36 | 1.11 | 1.19 | 1.15 | 1.36 |
| 1.35 | 1.10 | 1.18 | 1.14 | 1.35 |
| 1.34 | 1.09 | 1.17 | 1.13 | 1.34 |
| 1.33 | 1.08 | 1.16 | 1.12 | 1.33 |
| 1.32 | 1.07 | 1.15 | 1.11 | 1.32 |
| 1.31 | 1.06 | 1.14 | 1.10 | 1.31 |
| 1.30 | 1.05 | 1.13 | 1.09 | 1.30 |
| 1.29 | 1.04 | 1.12 | 1.08 | 1.29 |
| 1.28 | 1.03 | 1.11 | 1.07 | 1.28 |
| 1.27 | 1.02 | 1.10 | 1.06 | 1.27 |
| 1.26 | 1.01 | 1.09 | 1.05 | 1.26 |
| 1.25 | 1.00 | 1.08 | 1.04 | 1.25 |
| 1.24 | | 1.07 | 1.03 | 1.24 |
| 1.23 | | 1.06 | 1.02 | 1.23 |
| 1.22 | | 1.06 | 1.01 | 1.22 |
| 1.21 | | 1.04 | 1.00 | 1.21 |
| 1.20 | | 1.03 | | 1.20 |
| 1.19 | | 1.02 | | 1.19 |
| 1.18 | | 1.01 | | 1.18 |
| 1.17 | | 1.00 | | 1.17 |
| 1.16 | | | | 1.16 |

The reference value is the A value (the averaged ankle/wrist quotient) and all of the B, C, D and E values along this horizontal line are the norms for the individual. Reference values above this horizontal reference line represent hypo functioning glands and values below this line represent hyper functioning glands. If the individual is male, reference values of 1.48 in the A and E columns is considered normal. Reference values higher than 1.48 are more gynic. Reference values below 1.48 are more andric. In the event the individual is female and the reference values of 1.55 in the A and E columns is considered normal. Values higher than 1.55 are more gynic. Values lower than 1.55 are more andric. This analysis is an approximation based on body measurements and errors can occur from incorrect measuring. If values are very close to the considered normal values there may still be a slight hyper or hypo function and andricity or gynicity may be questionable. In the past, Dr. Page identified hereinbefore asserted that the B, C, and D reference values are about 95% accurate in determining the activity of the pituitary and thyroid glands and the reference values A and E are about 80% accurate for determining andricity and gynicity. In this case it may be necessary to give one sex hormone for two to three days and then determine the calcium to phosphors ratio by a blood test to establish the andricitry or gynicity of the individual. You can also use the following body characteristics listed in Dr. Page's book Body Chemistry in Health and Disease as an aid to deciding andricity and gynicity. These characteristics are as follows:
1. andric—will have the back of his hands forward when the arms hang loosely at the sides.
2. gynic—shows more of the palm of his hands.
3. andric—may have a space between his thighs when the knees are together. There will be no space in the andric if he is also hypo-pituitary.
4. gynic—is more likely to have space between the thighs.
5. andric—pubic hairline is a triangle with the apex upward. There may be hair up to the navel.
6. gynics—especially women have the base of the triangle upward making a straight line.
7. andric women—may have a curved base of the triangle, the convex line toward the navel.
8. andric chin—slopes downward from the near point of the chin to the neck.
9. gynic chin—classic Greek chin and extends straight backward.
10. under active posterior pituitary women have a double curve on the hips.
   It is possible to start out andric in the A column and to be more gynic in the E column and vice versa. If this is so, the indicator on the A line is the probable true indicator of andricity of gynicity. For the rest of the glandular determinations the reference line on the anthropometric analysis table is the same line as the reference value A.

The reference values A through E established for an individual are found in the Anthropometric Analysis Table and the values circled or otherwise marked. The Anthropometric Analysis Table may be embedded in a computer program designed to allow the user to insert reference values as the quotients derived as a function entries of circumferential measurements to the computer program. The method of self-measurement can be incorporated into a computer program in a manner such that the computer program computes the quotients required in the system and plots a graph or graphs and/or marks or computes a numerical table showing the inherited glandular pattern indicated by such measurements. Inputs to the computer program include self-measurement or measurement using a measuring tape and used to plot a graph or graphs and/or marks or compute a numerical table showing the inherited glandular pattern indicated by such measurements and indicates the disease tendencies and physical and mental characteristics associated with such a pattern. Additionally, the program is used to determine treatment sequence as well as the proper treatment substances using glandular supplements, synthetic posterior pituitary gland, and hormones. A computer analysis of the measured and reference values is within the skill of one skilled in the art. The graph uses of a saw tooth waveform to display the reference values is then used to plot the first plotting reference value A and then using this point to continue the plotting of the reference values on the graph. The plot line from A to B should be parallel to the other reference lines on the graph only when it represents the norm for the patient. The remaining plot lines are constructed parallel to the other reference lines on the graph. The use of a Table or a graph provides a visual basis for assessing deviations from the norm and the basis for an interpretation analysis as follows:
1. Anywhere the graph line is above the reference line it represents under activity of that gland.
2. If the graph line is below the reference line, it represents over activity of the gland.
3. Ideal sex hormone balance for females is at the “F” level on the A line, above this is more gynic, below is more andric.
4. Ideal sex hormone balance for males is at the “M” level on the A line, above this is more gynic, below this is more andric. Most people are either andric or gynic.
5. Sex hormone determinations using the graph or table are about 80% accurate. The other glandular determinations are 95-98% accurate.

Example 1, Joe Feelgood established five sites for circumferential length measurement along each of his right and left forearm and lower leg, then preformed the ordered sets of five self circumferential measurements followed by calculating an ordered set of five ratios comprising the quotient of each successive lower leg circumferential length measurement divided by the respective forearm circumferential length measurement for each circumferential length measurement at the quarter points. The averaged quotients are given as follows:

Averaged Quotient Chart for Joe Feelgood

| | | | | |
|---|---|---|---|---|
| Quotient Right | Quotient Right | Quotient Right | Quotient Right | Quotient Right |
| Wrist Divided | ¼ Arm Divided | ½ Arm Divided | ¾ Arm Divided | Elbow Divided |
| By Right Leg | By Right ¼ Leg | By Right ½ Leg | By Right ¾ Leg | By Right Knee |
| 1.42 (A Value) | 1.38 (B Value) | 1.48 (C Value) | 1.40 (D Value) | 1.40 (E Value) |

Applying the averaged ordered set of actual quotient values to obtain an evaluation based on the given reference values for a male produces a display as follows:

Anthropometric Analysis Table (truncated) for Joe Feelgood

| | | Posterior | Anterior | |
|---|---|---|---|---|
| Sex | Thyroid | Pituitary | Pituitary | Sex |
| Hormones | Gland | Gland | Gland | Hormones |
| A Values | B Values | C Values | D Values | E Values |
| 1.70 | 1.45 | 1.53 | 1.49 | 1.70 |
| 1.69 | 1.44 | 1.52 | 1.48 | 1.69 |
| 1.68 | 1.43 | 1.51 | 1.47 | 1.68 |
| 1.67 | 1.42 | 1.50 | 1.46 | 1.67 |
| 1.66 | 1.41 | 1.49 | 1.45 | 1.66 |
| 1.65 | 1.40 | 1.48 | 1.44 | 1.65 |
| 1.64 | 1.39 | 1.47 | 1.43 | 1.64 |
| 1.63 | 1.38 | 1.46 | 1.42 | 1.63 |
| 1.62 | 1.37 | 1.45 | 1.41 | 1.62 |
| 1.61 | 1.36 | 1.44 | 1.40 | 1.61 |
| 1.60 | 1.35 | 1.43 | 1.39 | 1.60 |
| 1.59 | 1.34 | 1.42 | 1.38 | 1.59 |
| 1.58 | 1.33 | 1.41 | 1.37 | 1.58 |
| 1.57 | 1.32 | 1.40 | 1.36 | 1.57 |
| 1.56 | 1.31 | 1.39 | 1.35 | 1.56 |
| 1.55 (female | 1.30 | 1.38 | 1.34 | 1.55 |
| reference | | | | |
| point) | | | | |
| 1.54 | 1.29 | 1.37 | 1.33 | 1.54 |
| 1.53 | 1.28 | 1.36 | 1.32 | 1.53 |
| 1.52 | 1.27 | 1.35 | 1.31 | 1.52 |
| 1.51 | 1.26 | 1.34 | 1.30 | 1.51 |
| 1.50 | 1.25 | 1.33 | 1.29 | 1.50 |
| 1.49 | 1.24 | 1.32 | 1.28 | 1.49 |
| 1.48 (male | 1.23 | 1.31 | 1.27 | 1.48 |
| reference | | | | |
| point) | | | | |
| 1.47 | 1.22 | 1.30 | 1.26 | 1.47 |
| 1.46 | 1.21 | 1.29 | 1.25 | 1.46 |
| 1.45 | 1.20 | 1.28 | 1.24 | 1.45 |
| 1.44 | 1.19 | 1.27 | 1.23 | 1.44 |
| 1.43 | 1.18 | 1.26 | 1.22 | 1.43 |
| 1.42 | 1.17 | 1.25 | 1.21 | 1.42 |
| 1.41 | 1.16 | 1.24 | 1.20 | 1.41 |
| 1.40 | 1.15 | 1.23 | 1.19 | 1.40 |
| 1.39 | 1.14 | 1.22 | 1.18 | 1.39 |
| 1.38 | 1.13 | 1.21 | 1.17 | 1.38 |
| 1.37 | 1.12 | 1.20 | 1.16 | 1.37 |
| 1.36 | 1.11 | 1.19 | 1.15 | 1.36 |
| 1.35 | 1.10 | 1.18 | 1.14 | 1.35 |

An evaluation of the Anthropometric Analysis Table reveals that Joe Feelgood is andric, hypothyroid, hypo-posterior pituitary, hypo-anterior pituitary.

Case history 1,a female patient age thirty as previously reported by Dr. Page was found with posterior pituitary gland deficiency, periodontal disease, and diabetes insipidus using the criteria given hereinbefore but without the benefit of the Anthropometric Analysis Table of the present invention. As reported, on a basic controlled diet her serum calcium was 9.4, serum phosphorous was 4.1, and blood sugar was 286. This is typical with this deficiency where the serum phosphorous is high relative to the calcium. Such a ratio leads to bone loss typical in periodontal disease, osteoporosis, and some forms of arthritis. Part of her diabetic problem is diabetes insipidus related to posterior pituitary gland deficiency. Ordered sets of limb measurement on the right and left sides for the arms and lower legs are recorded and the quotients are computed as follows:

Circumferential Measurement and Quotient Chart for Case history 1

| Right side | | | Left side | | |
|---|---|---|---|---|---|
| Wrist | Ankle | Quotient | Wrist | Ankle | Quotient |
| Circumference | Circumference | Ankle Divided | Circumference | Circumference | Ankle Divided |
| 5.65 | 9.2 | By Wrist | 5.65 | 9.2 | By Wrist |
| | | 1.62 (A Value) | | | 1.62 (A Value) |
| ¼ Arm | ¼ Leg | Quotient | ¼ Arm | ¼ Leg | Quotient |
| Circumference | Circumference | ¼ leg divided | Circumference | Circumference | ¼ leg divided |
| 5.95 | 9.3 | by ¼ arm | 5.65 | 9.15 | by ¼ arm |
| | | 1.56 (B Value) | | | 1.61 (B Value) |
| ½ Arm | ½ Leg | Quotient | ½ Arm | ½ Leg | Quotient |
| Circumference | Circumference | ½ leg divided | Circumference | Circumference | ½ divided by |
| 7.1 | 12.5 | by ½ arm | 6.7 | 12.65 | ½ arm |
| | | 1.76 (C Value) | | | 1.88 (C Value) |
| ¾ Arm | ¾ Leg | Quotient | ¾ Arm | ¾ Leg | Quotient |
| Circumference | Circumference | ¾ leg divided | Circumference | Circumference | ¾ leg divided |
| 8 | 11.15 | by ¾ arm | 7.7 | 11.35 | by ¾ arm |
| | | 1.39 (A Value) | | | 1.47 (A Value) |
| Elbow | Knee | Quotient | Elbow | Knee | Quotient |
| Circumference | Circumference | Knee divided | Circumference | Circumference | Knee divided |
| 8.3 | 12.9 | by Elbow | 8.05 | 13.0 | by Elbow |
| | | 1.56 (E Value) | | | 1.61 (E Value) |

Using the quotient portion of the chart, the ordered set of values is recorded as follows:

Averaged Quotient Chart for case history 1

| | | | | |
|---|---|---|---|---|
| Quotient Right | Quotient Right | Quotient Right | Quotient Right | Quotient Right |
| Wrist and Leg | ¼ Arm and Leg | ½ Arm and Leg | ¾ Arm and Leg | Elbow and Knee |
| Plus Quotient | Plus Quotient | Plus Quotient | Plus Quotient | Plus Quotient Left |
| Left | Left ¼ Arm and | Left ½ Arm and | Left ¾ Arm and | Elbow and Knee |
| Wrist and Leg | Leg Divided By | Leg Divided By | Leg Divided By | Divided By |
| Divided By 2 = 1.62 | 2 = 1.59 | 2 = 1.83 | 2 = 1.43 | 2 = 1.59 |
| (A Value) | (B Value) | (C Value) | (D Value) | (E Value) |

Using the Anthropometric Analysis Table of the present invention provides a quickly comprehensive visual comparison of the ordered set of actual values with the reference values for a female as follows:

Anthropometric Analysis Table (truncated) for Case history 1

| | | Posterior | Anterior | |
|---|---|---|---|---|
| Sex | Thyroid | Pituitary | Pituitary | Sex |
| Hormones | Gland | Gland | Gland | Hormones |
| A Values | B Values | C Values | D Values | E Values |
| 2.05 | 1.80 | 1.88 | 1.84 | 2.05 |
| 2.04 | 1.79 | 1.87 | 1.83 | 2.04 |
| 2.03 | 1.78 | 1.86 | 1.82 | 2.03 |
| 2.02 | 1.77 | 1.85 | 1.81 | 2.02 |
| 2.01 | 1.76 | 1.84 | 1.80 | 2.01 |
| 2.00 | 1.75 | 1.83 | 1.79 | 2.00 |
| 1.99 | 1.74 | 1.82 | 1.78 | 1.99 |
| 1.98 | 1.73 | 1.81 | 1.77 | 1.98 |
| 1.97 | 1.72 | 1.80 | 1.76 | 1.97 |
| 1.96 | 1.71 | 1.79 | 1.75 | 1.96 |
| 1.95 | 1.70 | 1.78 | 1.74 | 1.95 |
| 1.94 | 1.69 | 1.77 | 1.73 | 1.94 |
| 1.93 | 1.68 | 1.76 | 1.72 | 1.93 |
| 1.92 | 1.67 | 1.75 | 1.71 | 1.92 |
| 1.91 | 1.66 | 1.74 | 1.70 | 1.91 |
| 1.90 | 1.65 | 1.73 | 1.69 | 1.90 |
| 1.89 | 1.64 | 1.72 | 1.68 | 1.89 |
| 1.88 | 1.63 | 1.71 | 1.67 | 1.88 |
| 1.87 | 1.62 | 1.70 | 1.66 | 1.87 |
| 1.86 | 1.61 | 1.69 | 1.65 | 1.86 |
| 1.85 | 1.60 | 1.68 | 1.64 | 1.85 |
| 1.84 | 1.59 | 1.67 | 1.63 | 1.84 |
| 1.83 | 1.58 | 1.66 | 1.62 | 1.83 |
| 1.82 | 1.57 | 1.65 | 1.61 | 1.82 |
| 1.81 | 1.56 | 1.64 | 1.60 | 1.81 |
| 1.80 | 1.55 | 1.63 | 1.59 | 1.80 |
| 1.79 | 1.54 | 1.62 | 1.58 | 1.79 |
| 1.78 | 1.53 | 1.61 | 1.57 | 1.78 |
| 1.77 | 1.52 | 1.60 | 1.56 | 1.77 |
| 1.76 | 1.51 | 1.59 | 1.55 | 1.76 |
| 1.75 | 1.50 | 1.58 | 1.54 | 1.75 |
| 1.74 | 1.49 | 1.57 | 1.53 | 1.74 |
| 1.73 | 1.48 | 1.56 | 1.52 | 1.73 |
| 1.72 | 1.47 | 1.55 | 1.51 | 1.72 |
| 1.71 | 1.46 | 1.54 | 1.50 | 1.71 |
| 1.70 | 1.45 | 1.53 | 1.49 | 1.70 |
| 1.69 | 1.44 | 1.52 | 1.48 | 1.69 |
| 1.68 | 1.43 | 1.51 | 1.47 | 1.68 |
| 1.67 | 1.42 | 1.50 | 1.46 | 1.67 |
| 1.66 | 1.41 | 1.49 | 1.45 | 1.66 |
| 1.65 | 1.40 | 1.48 | 1.44 | 1.65 |
| 1.64 | 1.39 | 1.47 | 1.43 | 1.64 |
| 1.63 | 1.38 | 1.46 | 1.42 | 1.63 |
| 1.62 | 1.37 | 1.45 | 1.41 | 1.62 |
| 1.61 | 1.36 | 1.44 | 1.40 | 1.61 |
| 1.60 | 1.35 | 1.43 | 1.39 | 1.60 |
| 1.59 | 1.34 | 1.42 | 1.38 | 1.59 |
| 1.58 | 1.33 | 1.41 | 1.37 | 1.58 |
| 1.57 | 1.32 | 1.40 | 1.36 | 1.57 |
| 1.56 | 1.31 | 1.39 | 1.35 | 1.56 |
| 1.55 (female | 1.30 | 1.38 | 1.34 | 1.55 |
| reference | | | | |
| point) | | | | |
| 1.54 | 1.29 | 1.37 | 1.33 | 1.54 |
| 1.53 | 1.28 | 1.36 | 1.32 | 1.53 |
| 1.52 | 1.27 | 1.35 | 1.31 | 1.52 |
| 1.51 | 1.26 | 1.34 | 1.30 | 1.51 |
| 1.50 | 1.25 | 1.33 | 1.29 | 1.50 |

The quotient values compared with the reference values in the Anthropometric Analysis Table produce findings are she is deficient in posterior pituitary and thyroid. She was given one grain of thyroid and 6/100 mg of posterior pituitary. Her calcium/phosphorous ratio improved to 9.4 calcium and phosphorous at 3.3. Her blood sugar improved somewhat to 260 after one week of treatment. This patient was not checked long term because she voluntarily discontinued treatment. She was seen fifteen years later and reported that her internist reported that she had lost at least one inch in height over period of five years. She is receiving allopathic treatment for diabetes and osteoporosis.

Case history 2, a fourteen-year-old boy presented after a suicide attempt with Tylenol. His diagnosis was clinical depression due to a negative life event but was not prescribed antidepressants due to residual liver damage from the Tylenol. Ordered sets of limb measurement on the right and left sides for the arms and lower legs are recorded and the quotients are computed as follows:

Circumferential Measurement and Quotient Chart for Case history 2

| Right side | | | Left side | | |
|---|---|---|---|---|---|
| Wrist | Ankle | Quotient | Wrist | Ankle | Quotient |
| Circumference | Circumference | Ankle Divided | Circumference | Circumference | Ankle Divided |
| 6.9 | 9.8 | By Wrist | 7.0 | 9.6 | By Wrist |
| | | 1.42 (A Value) | | | 1.37 (A Value) |
| ¼ Arm | ¼ Leg | Quotient | ¼ Arm | ¼ Leg | Quotient |
| Circumference | Circumference | ¼ leg divided | Circumference | Circumference | ¼ leg divided |
| 7.1 | 10.0 | by ¼ arm | 7.2 | 9.6 | by ¼ arm |
| | | 1.41 (B Value) | | | 1.37 (B Value) |
| ½ Arm | ½ Leg | Quotient | ½ Arm | ½ Leg | Quotient |
| Circumference | Circumference | ½ leg divided | Circumference | Circumference | ½ divided by |
| 8.5 | 13.8 | by ½ arm | 8.5 | 13.1 | ½ arm |
| | | 1.60 (C Value) | | | 1.54 (C Value) |
| ¾ Arm | ¾ Leg | Quotient | ¾ Arm | ¾ Leg | Quotient |
| Circumference | Circumference | ¾ leg divided | Circumference | Circumference | ¾ leg divided |
| 9.6 | 12.9 | by ¾ arm | 9.6 | 13.0 | by ¾ arm |
| | | 1.34 (A Value) | | | 1.35 (A Value) |
| Elbow | Knee | Quotient | Elbow | Knee | Quotient |
| Circumference | Circumference | Knee divided | Circumference | Circumference | Knee divided |
| 10.5 | 15.0 | by Elbow | 10.6 | 15.1 | by Elbow |
| | | 1.43 (E Value) | | | 1.42 (E Value) |

Using the quotient portion of the chart, the ordered set of averaged values is recorded as follows:

Averaged Quotient Chart for case history 2

| | | | | |
|---|---|---|---|---|
| Quotient Right | Quotient Right | Quotient Right | Quotient Right | Quotient Right |
| Wrist and Leg | ¼ Arm and Leg | ½ Arm and Leg | ¾ Arm and Leg | Elbow and |
| Plus Quotient | Plus Quotient | Plus Quotient | Plus Quotient | Knee Plus |
| Left | Left ¼ Arm and | Left ½ Arm and | Left ¾ Arm and | Quotient Left |
| Wrist and Leg | Leg Divided By | Leg Divided By | Leg Divided By | Elbow and |
| Divided By 2 = | 2 = | 2 = | 2 = | Knee Divided |
| 1.39(A Value) | 1.39(B Value) | 1.57(C Value) | 1.34(D Value) | By 2 = |
| | | | | 1.43(E Value) |

Using the Anthropometric Analysis Table of the present invention the visual comparison of the ordered set of actual values with the reference values for a male as follows:

Anthropometric Analysis Table (truncated) for case history 2

| | | Posterior | Anterior | |
|---|---|---|---|---|
| Sex | Thyroid | Pituitary | Pituitary | Sex |
| Hormones | Gland | Gland | Gland | Hormones |
| A Values | B Values | C Values | D Values | E Values |
| 1.75 | 1.50 | 1.58 | 1.54 | 1.75 |
| 1.74 | 1.49 | 1.57 | 1.53 | 1.74 |
| 1.73 | 1.48 | 1.56 | 1.52 | 1.73 |
| 1.72 | 1.47 | 1.55 | 1.51 | 1.72 |
| 1.71 | 1.46 | 1.54 | 1.50 | 1.71 |
| 1.70 | 1.45 | 1.53 | 1.49 | 1.70 |
| 1.69 | 1.44 | 1.52 | 1.48 | 1.69 |
| 1.68 | 1.43 | 1.51 | 1.47 | 1.68 |
| 1.67 | 1.42 | 1.50 | 1.46 | 1.67 |
| 1.66 | 1.41 | 1.49 | 1.45 | 1.66 |
| 1.65 | 1.40 | 1.48 | 1.44 | 1.65 |
| 1.64 | 1.39 | 1.47 | 1.43 | 1.64 |
| 1.63 | 1.38 | 1.46 | 1.42 | 1.63 |
| 1.62 | 1.37 | 1.45 | 1.41 | 1.62 |
| 1.61 | 1.36 | 1.44 | 1.40 | 1.61 |
| 1.60 | 1.35 | 1.43 | 1.39 | 1.60 |
| 1.59 | 1.34 | 1.42 | 1.38 | 1.59 |
| 1.58 | 1.33 | 1.41 | 1.37 | 1.58 |
| 1.57 | 1.32 | 1.40 | 1.36 | 1.57 |
| 1.56 | 1.31 | 1.39 | 1.35 | 1.56 |
| 1.55 (female | 1.30 | 1.38 | 1.34 | 1.55 |
| reference | | | | |
| point) | | | | |
| 1.54 | 1.29 | 1.37 | 1.33 | 1.54 |
| 1.53 | 1.28 | 1.36 | 1.32 | 1.53 |
| 1.52 | 1.27 | 1.35 | 1.31 | 1.52 |
| 1.51 | 1.26 | 1.34 | 1.30 | 1.51 |
| 1.50 | 1.25 | 1.33 | 1.29 | 1.50 |
| 1.49 | 1.24 | 1.32 | 1.28 | 1.49 |
| 1.48 (male | 1.23 | 1.31 | 1.27 | 1.48 |
| reference | | | | |
| point) | | | | |
| 1.47 | 1.22 | 1.30 | 1.26 | 1.47 |
| 1.46 | 1.21 | 1.29 | 1.25 | 1.46 |
| 1.45 | 1.20 | 1.28 | 1.24 | 1.45 |
| 1.44 | 1.19 | 1.27 | 1.23 | 1.44 |
| 1.43 | 1.18 | 1.26 | 1.22 | 1.43 |
| 1.42 | 1.17 | 1.25 | 1.21 | 1.42 |
| 1.41 | 1.16 | 1.24 | 1.20 | 1.41 |
| 1.40 | 1.15 | 1.23 | 1.19 | 1.40 |
| 1.39 | 1.14 | 1.22 | 1.18 | 1.39 |
| 1.38 | 1.13 | 1.21 | 1.17 | 1.38 |
| 1.37 | 1.12 | 1.20 | 1.16 | 1.37 |
| 1.36 | 1.11 | 1.19 | 1.15 | 1.36 |
| 1.35 | 1.10 | 1.18 | 1.14 | 1.35 |

These measurements and quotient calculations reveal the boy is found to be very deficient in posterior pituitary and deficient in anterior pituitary. The glandular pattern of the boy as revealed by the Anthropometric Analysis Table produces an initial diagnosis of clinical depression resolved as actually a manic-depressive because of the typical glandular pattern of a manic-depressive. On the basic controlled diet, his calcium was 9.5, phosphorous was 4.3, and his blood sugar was 111. The phosphorous is high relative to the calcium. He had chronic inflamed gums, joint problems predominately as knee pain. His blood sugar was slightly high. The boy was treated with posterior and anterior pituitary synthetic glandular substance. He was given 1/400 mg of whole pituitary and 1/400 mg of posterior pituitary. His calcium/phosphorous ratio normalized to calcium 10.0, phosphorous 3.9, and blood sugar 86. His gum condition immediately cleared up with no localized dental treatment and his mood improved the next day after starting treatment. Subsequent personality questionnaires revealed no subsequent depression and there were no breakthrough episodes of mania or depression in a two-year follow-up. He takes his glandular supplements every day.

Case history 3 is of female patient age 59, a manic-depressive since age 17 and takes at least five prescription medications, not all related to manic depression. One ordered set of limb measurements on the left side for the arms and lower legs is recorded and the quotients are computed as follows:

Circumferential Measurement and Quotient Chart for
Case history 3 Left side

| | | |
|---|---|---|
| Wrist | Ankle | Quotient |
| Circumference | Circumference | Ankle Divided |
| 6.3 | 9.1 | By Wrist |
| | | 1.44 (A Value) |
| ¼ Arm | ¼ Leg | Quotient |
| Circumference | Circumference | ¼ leg divided |
| 7.5 | 9.1 | by ¼ arm |
| | | 1.21 (B Value) |
| ½ Arm | ½ Leg | Quotient |
| Circumference | Circumference | ½ divided by |
| 8.6 | 14.0 | ½ arm |
| | | 1.63 (C Value) |
| ¾ Arm | ¾ Leg | Quotient |
| Circumference | Circumference | ¾ leg divided |
| 10.5 | 14.1 | by ¾ arm |
| | | 1.34 (D Value) |
| Elbow | Knee | Quotient |
| Circumference | Circumference | Knee divided |
| 11.0 | 15.3 | by Elbow |
| | | 1.39 (E Value) |

Using the Anthropometric Analysis Table of the present invention the visual comparison of the ordered set of actual values with the reference values for the female of case history 3 as follows:

Anthropometric Analysis Table (truncated) for case history 3

| | | Posterior | Anterior | |
|---|---|---|---|---|
| Sex | Thyroid | Pituitary | Pituitary | Sex |
| Hormones | Gland | Gland | Gland | Hormones |
| A Values | B Values | C Values | D Values | E Values |
| 1.85 | 1.60 | 1.68 | 1.64 | 1.85 |
| 1.84 | 1.59 | 1.67 | 1.63 | 1.84 |
| 1.83 | 1.58 | 1.66 | 1.62 | 1.83 |
| 1.82 | 1.57 | 1.65 | 1.61 | 1.82 |
| 1.81 | 1.56 | 1.64 | 1.60 | 1.81 |
| 1.80 | 1.55 | 1.63 | 1.59 | 1.80 |
| 1.79 | 1.54 | 1.62 | 1.58 | 1.79 |
| 1.78 | 1.53 | 1.61 | 1.57 | 1.78 |
| 1.77 | 1.52 | 1.60 | 1.56 | 1.77 |
| 1.76 | 1.51 | 1.59 | 1.55 | 1.76 |
| 1.75 | 1.50 | 1.58 | 1.54 | 1.75 |
| 1.74 | 1.49 | 1.57 | 1.53 | 1.74 |
| 1.73 | 1.48 | 1.56 | 1.52 | 1.73 |
| 1.72 | 1.47 | 1.55 | 1.51 | 1.72 |
| 1.71 | 1.46 | 1.54 | 1.50 | 1.71 |
| 1.70 | 1.45 | 1.53 | 1.49 | 1.70 |
| 1.69 | 1.44 | 1.52 | 1.48 | 1.69 |
| 1.68 | 1.43 | 1.51 | 1.47 | 1.68 |
| 1.67 | 1.42 | 1.50 | 1.46 | 1.67 |
| 1.66 | 1.41 | 1.49 | 1.45 | 1.66 |
| 1.65 | 1.40 | 1.48 | 1.44 | 1.65 |
| 1.64 | 1.39 | 1.47 | 1.43 | 1.64 |
| 1.63 | 1.38 | 1.46 | 1.42 | 1.63 |
| 1.62 | 1.37 | 1.45 | 1.41 | 1.62 |
| 1.61 | 1.36 | 1.44 | 1.40 | 1.61 |
| 1.60 | 1.35 | 1.43 | 1.39 | 1.60 |
| 1.59 | 1.34 | 1.42 | 1.38 | 1.59 |
| 1.58 | 1.33 | 1.41 | 1.37 | 1.58 |
| 1.57 | 1.32 | 1.40 | 1.36 | 1.57 |
| 1.56 | 1.31 | 1.39 | 1.35 | 1.56 |
| 1.55 | 1.30 | 1.38 | 1.34 | 1.55 |
| (female | | | | |
| reference | | | | |
| point) | | | | |
| 1.54 | 1.29 | 1.37 | 1.33 | 1.54 |
| 1.53 | 1.28 | 1.36 | 1.32 | 1.53 |
| 1.52 | 1.27 | 1.35 | 1.31 | 1.52 |
| 1.51 | 1.26 | 1.34 | 1.30 | 1.51 |
| 1.50 | 1.25 | 1.33 | 1.29 | 1.50 |
| 1.49 | 1.24 | 1.32 | 1.28 | 1.49 |
| 1.48 | 1.23 | 1.31 | 1.27 | 1.48 |
| (male | | | | |
| reference | | | | |
| point) | | | | |
| 1.47 | 1.22 | 1.30 | 1.26 | 1.47 |
| 1.46 | 1.21 | 1.29 | 1.25 | 1.46 |
| 1.45 | 1.20 | 1.28 | 1.24 | 1.45 |
| 1.44 | 1.19 | 1.27 | 1.23 | 1.44 |
| 1.43 | 1.18 | 1.26 | 1.22 | 1.43 |
| 1.42 | 1.17 | 1.25 | 1.21 | 1.42 |
| 1.41 | 1.16 | 1.24 | 1.20 | 1.41 |
| 1.40 | 1.15 | 1.23 | 1.19 | 1.40 |
| 1.39 | 1.14 | 1.22 | 1.18 | 1.39 |
| 1.38 | 1.13 | 1.21 | 1.17 | 1.38 |
| 1.37 | 1.12 | 1.20 | 1.16 | 1.37 |
| 1.36 | 1.11 | 1.19 | 1.15 | 1.36 |
| 1.35 | 1.10 | 1.18 | 1.14 | 1.35 |

The glandular pattern of the female patient produced the diagnosis as manic-depressive. This manic-depressive female patient takes at least five prescription medications, not all related to manic depression but all related to her glandular pattern. The measured values verses reference values displayed by the table show that she is andric, extremely hypo posterior pituitary, hypo anterior pituitary, the typical glandular pattern of a manic-depressive.

Case History 4, a 91-year-old female presented at age 60 with full dentures due to bone loss. She had slightly high blood pressure and has exhibited hypomania for a day or two at a time at various times throughout her life. Her calcium on the basic controlled diet was 8.8, her phosphorous was 3.6, and blood sugar was 90. This is a high phosphorous relative to calcium, a condition which leads to bone loss. She took her medication for only a short period and then discontinued it. She was seen 24 years later. She now takes blood pressure medication; she has had one knee replacement and needs another. She has a degenerated disc in her back and requires a laminectomy and spinal fusion. Ordered sets of limb measurement on the right and left sides for the arms and lower legs are recorded and the quotients are computed as follows:

Circumferential Measurement and Quotient Chart for Case history 4

| Right side | | | Left side | | |
|---|---|---|---|---|---|
| Wrist | Ankle | Quotient | Wrist | Ankle | Quotient |
| Circumference | Circumference | Ankle Divided | Circumference | Circumference | Ankle Divided |
| 6.4 | 9.8 | By Wrist | 6.3 | 10.2 | By Wrist |
| | | 1.53 | | | 1.62 |
| | | (A Value) | | | (A Value) |
| ¼ Arm | ¼ Leg | Quotient | ¼ Arm | ¼ Leg | Quotient |
| Circumference | Circumference | ¼ leg divided | Circumference | Circumference | ¼ leg divided |
| 7.3 | 8.8 | by ¼ arm | 7.0 | 9.3 | by ¼ arm |
| | | 1.20 | | | 1.33 |
| | | (B Value) | | | (BValue) |
| ½ Arm | ½ Leg | Quotient | ½ Arm | ½ Leg | Quotient |
| Circumference | Circumference | ½ leg divided | Circumference | Circumference | ½ divided by |
| 8.9 | 12.2 | by ½ arm | 8.6 | 13.3 | ½ arm |
| | | 1.37 | | | 1.55 |
| | | (C Value) | | | (C Value) |
| ¾ Arm | ¾ Leg | Quotient | ¾ Arm | ¾ Leg | Quotient |
| Circumference | Circumference | ¾ leg divided | Circumference | Circumference | ¾ leg divided |
| 10.0 | 14.0 | by ¾ arm | 9.7 | 14.1 | by ¾ arm |
| | | 1.40 | | | 1.45 |
| | | (D Value) | | | (D Value) |
| Elbow | Knee | Quotient | Elbow | Knee | Quotient |
| Circumference | Circumference | Knee divided | Circumference | Circumference | Knee divided |
| 10.6 | 16.4 | by Elbow | 10.2 | 16.6 | by Elbow |
| | | 1.57 | | | 1.63 |
| | | (E Value) | | | (E Value) |

Using the quotient portion of the chart, the ordered set of averaged values is recorded as follows:

Averaged Quotient Chart for case history 4

| | | | | |
|---|---|---|---|---|
| Quotient Right | Quotient Right | Quotient Right | Quotient Right | Quotient Right |
| Wrist and Leg | ¼ Arm and Leg | ½ Arm and Leg | ¾ Arm and Leg | Elbow and Knee |
| Plus Quotient | Plus Quotient | Plus Quotient | Plus Quotient | Plus Quotient Left |
| Left Wrist and | Left ¼ Arm and | Left ½ Arm and | Left ¾ Arm and | Elbow and Knee |
| Leg Divided By | Leg Divided By | Leg Divided By | Leg Divided By | Divided By |
| 2 = 1.58 | 2 = 1.27 | 2 = 1.46 | 2 = 1.43 | 2 = 1.60 |
| (A Value) | (B Value) | (C Value) | (D Value) | (E Value) |

Using the Anthropometric Analysis Table of the present invention the visual comparison of the ordered set of actual values with the reference values for a female as follows:

Anthropometric Analysis Table (truncated) for case history 4

| | | Posterior | Anterior | |
|---|---|---|---|---|
| Sex | Thyroid | Pituitary | Pituitary | Sex |
| Hormones | Gland | Gland | Gland | Hormones |
| A Values | B Values | C Values | D Values | E Values |
| 1.70 | 1.45 | 1.53 | 1.49 | 1.70 |
| 1.69 | 1.44 | 1.52 | 1.48 | 1.69 |
| 1.68 | 1.43 | 1.51 | 1.47 | 1.68 |
| 1.67 | 1.42 | 1.50 | 1.46 | 1.67 |
| 1.66 | 1.41 | 1.49 | 1.45 | 1.66 |
| 1.65 | 1.40 | 1.48 | 1.44 | 1.65 |
| 1.64 | 1.39 | 1.47 | 1.43 | 1.64 |
| 1.63 | 1.38 | 1.46 | 1.42 | 1.63 |
| 1.62 | 1.37 | 1.45 | 1.41 | 1.62 |
| 1.61 | 1.36 | 1.44 | 1.40 | 1.61 |
| 1.60 | 1.35 | 1.43 | 1.39 | 1.60 |
| 1.59 | 1.34 | 1.42 | 1.38 | 1.59 |
| 1.58 | 1.33 | 1.41 | 1.37 | 1.58 |
| 1.57 | 1.32 | 1.40 | 1.36 | 1.57 |
| 1.56 | 1.31 | 1.39 | 1.35 | 1.56 |
| 1.55 | 1.30 | 1.38 | 1.34 | 1.55 |
| (female | | | | |
| reference | | | | |
| point) | | | | |
| 1.54 | 1.29 | 1.37 | 1.33 | 1.54 |
| 1.53 | 1.28 | 1.36 | 1.32 | 1.53 |
| 1.52 | 1.27 | 1.35 | 1.31 | 1.52 |
| 1.51 | 1.26 | 1.34 | 1.30 | 1.51 |
| 1.50 | 1.25 | 1.33 | 1.29 | 1.50 |

Glandular pattern of this 91-year-old female with anterior and posterior pituitary deficiency and degenerative arthritis. The quotients derived from the measurements revealed by the table show the patient was found to be equally deficient in anterior and posterior pituitary. Her calcium on the basic controlled diet was 8.8, her phosphorous was 3.6, and blood sugar was 90. This high phosphorous relative to calcium is a condition, which leads to bone loss. She was given 1/400 mg of whole pituitary and her calcium/phosphorous ratio improved to calcium 8.8, phosphorous 3.13, and blood sugar 99, a much more positive calcium/phosphorous ratio. She took her medication for only a short period and then discontinued it. She was seen 24 years later. She now takes blood pressure medication; she has had one knee replacement and needs another. She has a degenerated disc in her back and requires a laminectomy and spinal fusion.

This invention also improves the differential diagnosis of various diseases. Diabetes insipidus often coexists with diabetes mellitus. Diabetes insipidus can also be a contributing factor in type II diabetes which is believed due to insulin resistance. Diabetes insipidus is due to an under functioning posterior pituitary gland. The measuring technique and table gives an indication of the degree of sub functioning of the posterior pituitary gland and its contributing effect in these diseases. When the posterior pituitary gland is treated in such cases, the overall result is better with lower and more stable blood sugar values. There exists no conclusive diagnostic test for manic depression. The diagnosis may incorrectly be called clinical depression and the mania may not be diagnosed or treated. Also a diagnosis of ADD (attention deficit disorder) or ADHD (attention deficit hyperactive disorder) may be made when in actuality the correct diagnosis may be manic depression. This misdiagnosis can result in giving stimulants as a treatment which can exacerbate the condition. The graph and table values can give a definitive diagnosis of manic depression. These patients have a severe under functioning of the posterior pituitary gland. They have a characteristic graph and table values indicating such hypo functioning.

The posterior pituitary gland consists of two active chemical substances which can also be produced synthetically in the exact same proportion that exists in the natural state in humans and cattle. They are oxytocin and vasopressin. Compounding these substances to form a synthetic version of posterior pituitary gland has the advantage over the natural source by providing consistency in dosage and freedom from impurities or contamination. This invention also consists of a synthetic substance to treat deficiency of the posterior pituitary gland much as thyroid supplementation is used. Its effect in the treatment of depression and manic depression is due to the feedback effect of the posterior pituitary gland on the adrenal cortex by keeping the adrenal cortex from over functioning and by supplementing deficient amounts of oxytocin and vesopressin. Sub function of the posterior pituitary gland can be indicated by low specific gravity of the urine, high blood pressure, elevated blood sugar levels and abnormal calcium/phosphorous ratios. This invention corrects sub functioning of the posterior pituitary gland. In the usual embodiment, this synthetic glandular substance is taken as an oral tablet. Various dosages are tried until the optimal dosage is determined. The optimal dosage is determined as the one which produces the most ideal calcium and phosphorous ratio on a controlled diet for three days after a twelve-hour fast and also which produces a specific gravity of the urine which most closely approaches 1.022, again on a diet free of caffeine, alcohol and medications. This combination typically exists in tablet form to be taken orally. An alternative would be to provide it in such form as to be administered sublingually, transdermally or as a nasal spray. The tablet should be available in various dosages equivalent to the natural form which exists at the dosages of 1/800 mg, 1/400 mg, 1/100 mg, and 3/100 mg. These dosages have been used clinically and produce the best results. This invention may alternately be supplied as two separate pills with separate synthetic chemicals in each pill. This invention may also include substances which may be found in the natural posterior pituitary gland which have yet to be isolated such as fat mobilizing substances and which are believed to exist. This invention provides consistency of dosage and freedom from any impurities and contamination.

While the present invention has been described in connection with the preferred embodiments of the various figures, it is to be understood that other similar embodiments may be used or modifications and additions may be made to the described embodiment for performing the same function of the present invention without deviating therefrom. Therefore, the present invention should not be limited to any single embodiment, but rather construed in breadth and scope in accordance with the recitation of the appended claims.

1. An anthropometric method of diagnosing endocrine activity of a subject, said method including the steps of:
using an adhesive bearing substrate to establish five sites for circumferential length measurement along each of a forearm and lower leg of a subject, each of said five sites being established by indicia on said substrate at successive quarter points commencing at the outer most site of each of said forearm and lower leg of a subject;
calculating an ordered set of five ratios comprising the quotient of each successive lower leg circumferential length measurement divided by the respective forearm circumferential length measurement for each circumferential length measurement at said quarter points;
comparing the numerical values of said ordered set of five ratios to numerical values of an ordered set of five reference standards to determine hypo, normal or hyper activity of each of the ordered set of endocrine activity consisting of sex hormones; thyroid gland; posterior pituitary gland; anterior pituitary gland; and sex hormones.

2. The method according to claim 1 wherein said ordered set of five reference standards is used to form a family of base line norms of varying magnitude to allow assessment of the magnitude of said hypo, normal or hyper activity.

3. The method according to claim 1 wherein said family of base line norms appears as an anthropometric analysis table.

4. The method according to claim 3 wherein said anthropometric analysis table includes columns of numerical values aligned in rows forming ordered sets of abnormal reference numerical values varying greater and less in magnitudes than said ordered set of five reference standards.

5. The method according to claim 3 wherein said anthropometric analysis table appears as a combination five component side by side graphs representing said ordered set of five reference standards plotting numerical graduations or hypo, normal and hyper value for each such reference standard.

6. The method according to claim 1 including the further step of selecting synthetic chemicals to form a synthetic posterior pituitary gland to treat said subject having said hypo activity of the posterior pituitary gland.

7. The method according to claim 1 wherein including the further step of selecting synthetic chemicals and natural glandular products to treat hypo hyperactivity activity of endocrine glands.

8. The method according to claim 6 wherein said synthetic chemicals are chosen to treat hypo activity of the posterior pituitary gland, sex hormones and other hormones coming from the adrenal cortex of the subject.

9. The method according to claim 6 including the further step of determining dosages of said synthetic chemicals based on monitoring specific gravity of urine of said subject.

10. The method according to claim 9 wherein said step of determining dosages of said synthetic chemicals consist of determining dosages of vasopressin.

11. The method according to claim 9 wherein said monitoring specific gravity of said subject includes adjusting dosages of said synthetic chemicals to produce specific gravity values approximating 1.022.

12. The method according to claim 6 including the further step of determining dosages of said synthetic chemicals based on monitoring serum calcium to phosphorous ratios of said subject.

13. The method according to claim 12 wherein said monitoring scrum calcium to phosphorous ratios of said subject includes adjusting dosages of said synthetic chemicals based on attaining a 2½:1 ratio.

14. The method according to claim 13 including the further step of determining dosages of said synthetic chemicals based on monitoring specific gravity of urine of said subject.

15. The method according to claim 14 wherein said monitoring specific gravity of said subject includes adjusting dosages of said synthetic chemicals to produce specific gravity values approximating 1.022.

16. The method according to claim 6 wherein said synthetic chemicals consist of synthetic oxytocin and synthetic vasopressin.

17. The method according to claim 6 wherein said synthetic chemicals consist of synthetic vasopressin.

18. The method according to claim 6 wherein said synthetic chemicals consist of synthetic oxytocin.

19. The method according to claim 1 wherein said step of using an adhesive bearing substrate includes providing a ruler adapted to be brought into circumferential contact with each of said forearm and lower leg of a subject at each of the established five sites for using indicia on said ruler to measure sad circumferential length measurements at each of said successive quarter points.

20. The method according to claim 19 wherein said ruler is formed with a generally flat face surface having said indicia thereon.

21. The method according to claim 1 wherein said five sites for circumferential length measurement along each of a forearm and lower leg of a subject are further defined to comprise: an arm circumference at the wrist level; an arm circumference at one fourth the distance from the wrist to the elbow; an arm circumference at one half the distance from the wrist to the elbow; an arm circumference at three fourths the distance from the wrist to the elbow; an arm circumference at the elbow; a lower leg circumference at an ankle level; a lower leg circumference at one fourth the distance from the ankle to the knee; lower leg circumference at a one half the distance ankle to the knee; a lower leg circumference at three fourths the distance from the ankle to the knee; and lower leg circumference at the knee circumference.

22. The method according to claim 1 wherein said step of using an adhesive bearing substrate to establish five sites for circumferential length measurement along each of a forearm and lower leg of a subject includes:
seating said subject in a chair with the subjects feet flat on the floor and the chosen arm bent slightly with the palm of the hand placed on the knee;
applying an elbow indicia mark to the center of the elbow and a wrist indicia mark to the center of the wrist at the site of a protruding bone at the outside of the arm;
arranging said substrate on the outside of the arm to span the distance between said elbow indicia mark and said wrist indicia mark;
recording said elbow indicia mark and said wrist indicia mark on said substrate;
using length graduations of a rule to establish and then apply indicia markings at each of four equal length sections between said elbow indicia mark and said wrist indicia mark along said substrate; and
adhesively attaching said substrate to a subject to span the distance between said elbow indicia mark and said wrist indicia mark to display said indicia markings of four equal length sections.

23. The method according to claim 1 wherein said step of using an adhesive bearing substrate to establish five sites for circumferential length measurement along each of a forearm and lower leg of a subject includes:
seating said subject in a chair with the subject's heel on the floor and the bottom flat surface of the foot at right angles to the long axis of the lower leg;
applying a lower leg indicia marks to the center of the knee cap and an ankle bone mark on the outside of the ankle in the middle of the protruding bone;
arranging said substrate on the outside of the lower leg to span the distance between said indicia mark at the center of the knee cap and said ankle bone mark on the outside of the ankle;
recording said lower leg indicia marks on said substrate;
using length graduations of a rule to establish and then apply lower leg indicia markings at each of four equal length sections along said substrate; and
adhesively attaching said substrate to a subject to span the distance between said lower leg indicia marks to display said indicia markings of four equal length sections.
